(19) United States
(12) Patent Application Publication
(10) Pub. No.: US 2025/0204802 A1
(43) Pub. Date: Jun. 26, 2025

(54) IN VIVO NON-INVASIVE INTRACRANIAL PRESSURE MONITORING DEVICE AND METHOD BASED ON MENINGEAL ABSORBANCE CHANGES

(71) Applicants: Tianjin University, Tianjin City (CN); Hebei Jinkang'an Medical Device Technology Co., Ltd, Shijiazhuang City (CN)

(72) Inventors: Xiuyun LIU, Tianjin City (CN); Dong MING, Tianjin City (CN); Min ZHOU, Tianjin City (CN); Haodong LI, Tianjin City (CN); Mengqi HE, Tianjin City (CN); Junjian KANG, Tianjin City (CN); Luochen LI, Shijiazhuang City (CN); Xiaoyi WANG, Shenzhen City (CN); Tao LI, Tianjin City (CN); Yangong CHAO, Tianjin City (CN); Meijun PANG, Tianjin City (CN); Tengchao YIN, Shijiazhuang City (CN)

(73) Assignees: Tianjin University, Tianjin City (CN); Hebei Jinkang'an Medical Device Technology Co., Ltd, Shijiazhuang City (CN)

(21) Appl. No.: 18/415,660

(22) Filed: Jan. 18, 2024

(30) Foreign Application Priority Data
Dec. 20, 2023 (CN) 2023117597964

(51) Int. Cl. A61B 5/03; A61B 5/00

(52) U.S. Cl. CPC A61B 5/031; A61B 5/0075; A61B 5/7203; A61B 5/7271; A61B 2562/0238

(57) ABSTRACT

The present innovation unveils a non-invasive in vivo intracranial pressure (ICP) monitoring device and method relying on meningeal absorbance changes. Comprising a signal excitation module, spectral data acquisition module, and data processing module, the device incorporates a laser and optical parametric oscillator in the signal excitation module. Optical path adjustments are facilitated by concave lenses and convex lens mirrors between the laser's output and the optical parametric oscillator's input. The optical parametric oscillator's output is equipped with a coaxial lens group and a fiber bundle. This approach ensures non-invasive ICP monitoring, providing ease of use, precision, reliability, and continuous dynamic monitoring, significantly reducing patient discomfort during ICP monitoring. The method offers an objective foundation for disease diagnosis, condition assessment, and the formulation of effective diagnosis and treatment strategies, showcasing substantial clinical potential.

### TECHNICAL FIELD

The present invention relates to the field of neuro-intensive care unit technology. In particular, it involves an in vivo non-invasive intracranial pressure monitoring device and method based on meningeal absorbance changes.

### BACKGROUND ART

Increased ICP is common in brain diseases such as craniocerebral trauma, intracranial infection, cerebrovascular disease, and brain tumor. It can compress brain tissue and even cause brain herniation, causing adverse consequences. Accurate monitoring of intracranial pressure changes, reasonable confirmation of intracranial pressure intervention cut-offs, and effective control of intracranial pressure have become the key to reducing the mortality rate and improving the prognosis of neurological function.

Invasive ICP monitoring is regarded as the gold standard for intracranial pressure monitoring due to its high accuracy and continuous monitoring. The main monitoring methods include ventricular, parenchymal, subdural, epidural, etc. However, the clinical application of invasive intracranial pressure monitoring is limited to a considerable extent due to procedure-related complications such as intracranial hemorrhage and infection, as well as factors such as high cost and zero-point drift. Therefore, the search for an accurate, reliable, inexpensive, and continuously monitored non-invasive ICP monitoring technology has become the current clinical work. In particular, the neuro intensive care unit (ICU) is an urgent problem to be addressed.

International scholars are conducting relevant research on non-invasive intracranial pressure monitoring, such as intraocular pressure measurement by tonometry, optic nerve sheath diameter (ONSD) measurement by ocular ultrasound, transcranial Doppler ultrasound (TCD), somatosensory evoked potentials (SEP), and flash visual evoked potentials (flash). visual evoked potential (FVEP) and electroencephalogram (EEG) techniques to analyze ICP. However, there are shortcomings such as large measurement error and unsustainability, and further research is needed.

### SUMMARY

The object of the present invention is to provide an in vivo non-invasive intracranial pressure monitoring device and method based on meningeal absorbance changes, which can monitor intracranial ICP under non-invasive conditions. Easy-to-use, accurate, reliable, infection-free, and continuous dynamic monitoring greatly reduces the pain of patients in ICP monitoring. It can also provide an objective basis for doctors to diagnose diseases, judge conditions, and formulate further diagnosis and treatment plans.

In order to achieve the above purpose, the invention provides an in vivo noninvasive intracranial pressure monitoring and method based on meningeal absorbance changes. The monitoring device includes a signal excitation module, a spectral data acquisition module, and a data processing module. The signal excitation module includes a laser and an optical parametric oscillator. The spectral data acquisition module includes a spectrometer and its supporting data acquisition software. Several mirrors are arranged between the output of the laser and the input of the optical parametric oscillator to adjust the optical path. The output end of the optical parametric oscillator is successively set up with a coaxial lens group and a fiber bundle, and the optical fiber beam outlet is connected to the monitoring object. The probe of the spectrometer is in contact with the monitoring object, and the probe is in the same plane as the optical fiber beam outlet.

An in vivo non-invasive intracranial pressure monitoring method based on changes in meningeal absorbance, including the following steps:
S1. Pre-treatment and fixation of monitoring objects;
S2. Detect background spectral intensity: Turn on the spectrometer and place the spectrometer probe at the irradiation site of the subject's skull. The spectrometer continuously records the background spectral intensity in real time for 7 min and obtains the background spectral intensity data that changes with wavelength and time.
S3. Detect the intensity of the incident laser: turn on the laser and preheat the optical parametric oscillator. The spectrometer probe is on the same straight line as the beam outlet. With 1 s as a cycle, the probe of the spectrometer is irradiated twice with a near-infrared pulsed laser of a certain wavelength in a cycle. Record the intensity of the incident laser for 10 cycles.
S4. Detecting the intensity of the transmitted laser: the spectrometer probe is in the same plane as the fiber-optic beam. The fiber-optic beam emitter illuminates the cranial irradiation site of the monitoring object with a near-infrared pulsed laser of a certain wavelength. Turn on the spectrometer and place the spectrometer probe on the cranial probe site of the monitoring object. The intensity data of the transmitted laser was collected every 0.1 s.
S5. Data processing and absorbance calculations.

Preferably, the pretreatment and fixation in the step S1 comprise: anesthesia, removing and disinfecting the hair in the middle part of the skull of the monitoring subject, the monitoring subject lying supine, the body position is straight, and the head is at the level of the body axis.

Preferably, the irradiation site is the midpoint of the anterior fontanelle.

Preferably, the detection site in the step S3 is: any point within the radius of 0˜3 cm with the irradiation site as the center of the circle. Preferably, the near-infrared pulsed laser of a certain wavelength is one of 700 nm, 725 nm, 750 nm, 775 nm, and 800 nm. Preferably, the near-infrared pulsed laser of a certain wavelength is 700 nm and 800 nm.

Preferably, the data processing and absorbance calculations in the step S5 comprise:
S5-1. Calculate the noise value. According to the background spectral intensity data obtained in step S2 with wavelength and time, the data of the first 2 min are rounded. According to the resolution of the spectrometer, the minimum value is found in the three columns of background spectral intensity data of a certain wavelength±resolution. The average of the three minimum values is taken as the noise value.
S5-2. Calculate the incident laser energy. The intensity of the incident laser detected in step S3 is subtracted from the noise values, respectively. The average value is calculated as the incident laser energy.
S5-3. Calculate the transmitted laser energy. The transmitted laser intensities detected in step S4 are subtracted from the noise values, respectively. The average value is calculated as the transmitted laser energy.
S5-4. The absorbance is calculated as follows:

$$A = {log}_{10} \frac{{\overline{I}}_{t} ( λ )}{{\overline{I}}_{o} ( λ )}$$

A is the absorbance, I̅ₜ̅(λ) denotes the energy of transmitted light, I̅ₒ̅(λ) denotes the energy of the incident laser.

Near-infrared light has very good penetration for biological tissues and body fluids. The relative energy change between reflected and incident light is related to the absorbance and reflectance at the incident interface. The brain consists of the skull, dura mater, arachnoid, perichondrium and brain parenchyma. The thickness of the meninges changes slightly due to changes in intracranial pressure. At the same time, the thickness of the meninges affects the absorption and attenuation of light. Therefore, when NIR light is transmitted through the skull, the light signals emitted from the surface of the tissue will carry information about the structure and thickness of the meninges. By analyzing the information carried by these light signals, the monitoring of intracranial pressure can be realized.

Accordingly, an in-vivo non-invasive intracranial pressure monitoring device and method of the present invention using the above-described structure based on changes in meningeal absorbance has the following technical effects:
(1) Compared with the traditional invasive intracranial pressure monitoring technology, the present invention adopts a non-invasive monitoring method. The invention can monitor the ICP of the patient in a non-invasive manner, which has the advantages of safety, low risk of infection, and continuous dynamic monitoring, etc. It can greatly reduce the pain of the patient during the monitoring of ICP. Patients' pain in the process of monitoring ICP can be greatly reduced;
(2) Compared with other existing and under-development non-invasive ICP monitoring techniques, this method has the advantages of low cost, high sensitivity, fast response time, and freedom from electromagnetic interference.

The technical solution of the present invention is described in further detail below by means of the accompanying drawings and embodiments.

### BRIEF DESCRIPTION OF THE DRAWINGS

In order to more clearly describe the technical solutions in the embodiments or prior art of the present invention. The accompanying drawings to be used in the description of the embodiments or prior art will be briefly described below. Obviously, the accompanying drawings in the following description are merely exemplary. To a person of ordinary skill in the art, other embodiments of the drawings may be obtained by extending the drawings according to the provided drawings without creative labor.

FIG. 1 shows a schematic diagram of the structure of the monitoring device of embodiment 1 of this invention.

FIG. 2 shows a flowchart of embodiment 1 of this invention.

FIG. 3 shows the curve of ICP values at different injection doses in embodiment 1 of this invention.

FIG. 4 is the light absorption curve under different laser wavelengths of embodiment 1 of this invention;

FIG. 5 shows the relationship between absorbance A and ICP at 700 nm and 800 nm for this Example 1 of the invention. (A) is the relationship between absorbance A and ICP at 700 nm, and (B) is the relationship between absorbance A and ICP at 800 nm.

FIG. 6 Analysis of the agreement between the fitted equation ICP values and the invasive side measured ICP values at 700 nm and 800 nm wavelengths for embodiment 1 of this invention; (A) is the consistency analysis between the ICP value of the fitting equation at 700 nm wavelength and the ICP value of invasive ventricular monitoring. (B) Partial analysis of the consistency between the ICP values of the fitting equation at 800 nm wavelength and the ICP values of invasive ventricular monitoring.

### DETAILED DESCRIPTION OF THE EMBODIMENTS

The technical scheme of this invention is further described hereinafter by means of the accompanying drawings and embodiments.

Unless otherwise defined, technical or scientific terms used in this invention shall have the ordinary meaning understood by persons having ordinary skill in the field to which the invention belongs.

### Embodiment 1

Intracranial pressure monitoring was performed in rats in vivo using an in vivo noninvasive intracranial pressure monitoring device based on changes in meningeal absorbance of the present invention and an invasive intracranial pressure monitoring device in the prior art. Rats were selected as male SD rats, weighing 510 g. Source: Beijing Viton Lihua Laboratory Animal Technology Co., Ltd.

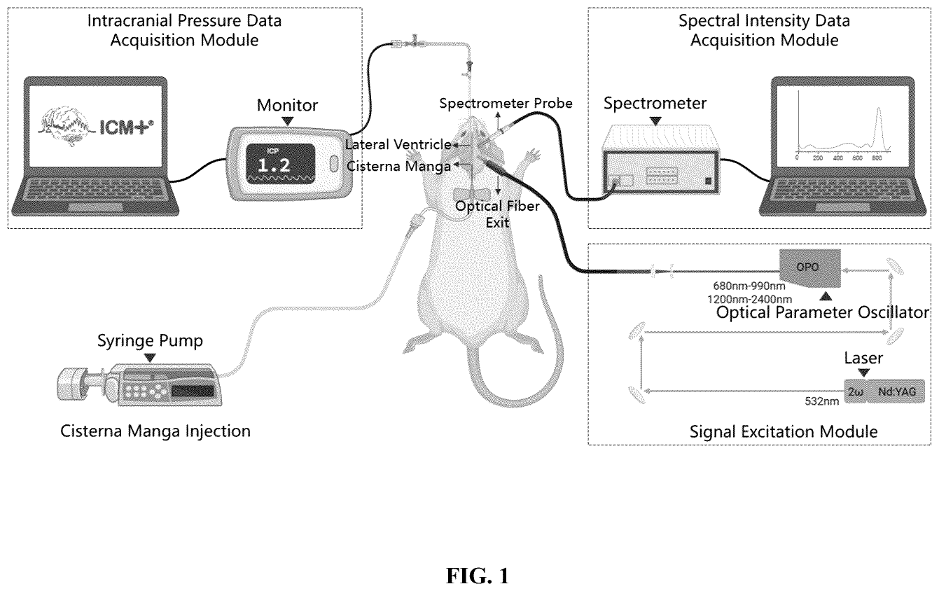

As shown in FIG. 1, it includes a signal excitation module, a spectral data acquisition module, an intracranial pressure data acquisition module, and a data processing module.

The signal excitation module is mainly based on a lamp-pumped pulsed Nd:YAG laser (Q-Smart 450) and an optical parametric oscillator (BB-OPO) for optical signal excitation.

The Nd:YAG laser acts as a pump source and is capable of emitting a fixed wavelength laser at 1064 nm. With the addition of a double frequency module, a fixed wavelength laser of 532 nm can be emitted. The laser energy can be controlled by special control software or touch screen.

A 532 nm laser (pump light) is passed into an optical parametric oscillator. The optical parametric oscillator also has a special control computer, control protocol and control software. By adjusting the set value in the control software, it is possible to generate laser light in the range of 680-990 nm (signal light) and 1200-2400 nm. This embodiment uses only 700 nm, 725 nm, 750 nm, 775 nm, 800 nm specific wavelength near-infrared pulsed lasers.

As shown in FIG. 1, concave lenses, convex lenses, and fiber bundles are also installed in the optical path construction part. The emitting end of the optical parametric oscillator is fixed with a coaxial lens group, and the other end of the lens group corresponds to a fiber bundle. The fiber bundle is used to convert the direction of laser irradiation and is 3 mm in diameter. The lens set is used to converge and then diverge the laser light emitted by the optical parametric oscillator so that the beam diameter matches the fiber bundle diameter. After the laser is emitted by the laser, the optical path is adjusted by the refraction of the concave lens and convex lens so that it passes into the optical parametric oscillator. The optical parametric oscillator then adjusts the beam diameter through a coaxial lens set so that the beam enters the fiber bundle. The optical fiber beam irradiates the skull irradiation site of the monitoring subject (with the fontanelle as a reference, 1.0 mm posteriorly and 1.5 mm to the right along the middle suture).

The spectral data acquisition module uses the ASC-UVNIR2 compact spectrometer to detect the original optical signal, and the JC spectrum software is used to display and save the data.

The intracranial pressure data acquisition module uses the GE Dash 4000 monitor, medical pressure sensor, and ICM+ multimodal monitoring software to monitor and record intracranial pressure data in real time. Medical pressure sensors are used to measure intracranial pressure, and monitors and ICM+ are used to monitor and record intracranial pressure data in real time.

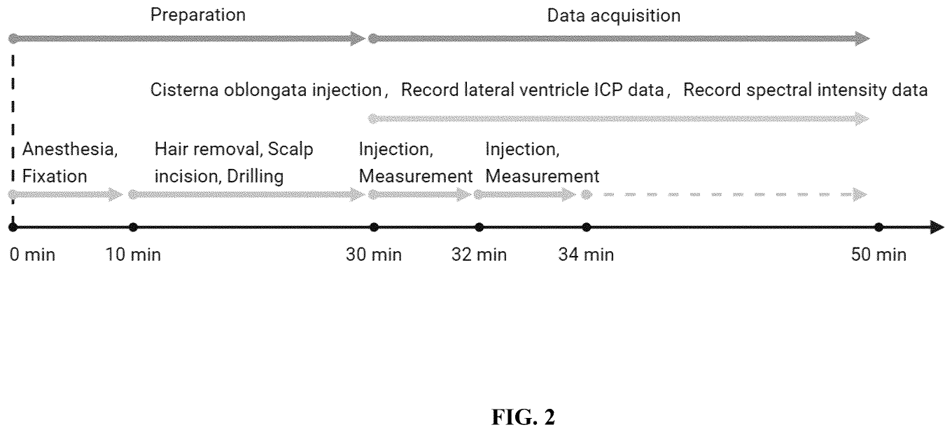

In vivo non-invasive intracranial pressure monitoring in rats is performed using the above devices. The process is shown in FIG. 2 and includes:
S1. Anesthesia: SD rats are anesthetized with 20% Uratan solution at an injection dose of 0.7 mL/100 g.
S2. Remove the hair from the middle part of the rat's skull to the cervical vertebrae, and wipe it with an alcohol cotton ball for disinfection. The anesthetized rat's entire skull is fixed horizontally on a brain stereotaxic instrument, and the head is elevated at 135° with the body.
S3, Invasive ventricular ICP monitoring: starting from the middle point of the two eyes of the rat and ending at 10 mm below the occipital crest. Cut everything along the axial axis of the rat's skull with a blade to expose the skull. The fontanelle was used as a reference 2.0 mm posteriorly and 1.5 mm to the left along the middle suture as a lateral ventricle monitoring ICP site.

Drill holes with a skull drill at the marked location. Be careful not to injure the meninges only through the skull. The needle of the medical sensor is inserted into the left lateral ventricle to a depth of 4.5 mm.
S4, cisterna manga injection: The occipital crest is found by touch, and the needle is inserted at the muscle space 3 mm below the occipital crest. The angle of needle insertion is parallel to the body. The needle is slowly advanced on the slope of the needle tip to the cisterna manga of the cerebellum to a depth of approximately 0.5 mm.
S5. Detect background spectral intensity: Turn on the spectrometer and place the spectrometer probe at the irradiation site (anterior fontanelle midpoint) of the rat skull. The spectrometer continuously recorded the background spectral intensity in real time for 7 min. Background spectral intensity data with wavelength and time were obtained.
S6. With 0.1 mL as the injection unit, normal saline is continuously injected into the cisterna manga of the cerebellum to cause a continuous increase in intracranial pressure, up to 1.8 mL. For every 0.1 mL injection, ICM+ software was used to record invasive intracranial pressure data in real time. And the optical signal intensity is detected by a spectrometer. The frame rate of the spectrometer is 50 fps, and the average of the results of 10 consecutive acquisitions is selected as the size of the detected optical signal.

Detection of light signal intensity includes:
S7. Detect the intensity of the incident laser: turn on the laser and preheat the optical parametric oscillator. The spectrometer probe is placed at the irradiation site of the rat skull (the midpoint of the fontanelle). The laser emits light sequentially from five wavelengths: 700 nm, 725 nm, 750 nm, 775 nm and 800 nm according to the coding order of the experiment. There is a cycle within 1 s, in which each wavelength of laser is emitted twice. Spectral data of laser luminescence were recorded for 10 cycles, and each wavelength of laser was emitted 20 times.
S8. Detect the intensity of the transmitted laser: keep the spectrometer probe in the same plane as the beam outlet. The optical end of the fiber beam irradiated the skull irradiation site of rats at five wavelengths of 700 nm, 725 nm, 750 nm, 775 nm, and 800 nm (the fontanelle was used as a reference, 1.0 mm backward and 1.5 mm to the right along the middle bone suture). Turn on the spectrometer and place the spectrometer probe at the skull probe site of the monitored object (use the fontanelle as a reference, 1.8 mm posteriorly and 1.5 mm to the right along the middle suture). The intensity data of the transmitted laser is collected every 0.1 s.

### Data Analysis

### (1) Data Processing for Invasive Intracranial Pressure Monitoring

ICM+ software and Python language are used to complete the processing of raw data. The following steps include data format conversion and data segmentation:
S1. Data format conversion

Using ICM+ software, the raw ICP monitoring data is converted into a common “.csv” format.
S2. Data segmentation

This system defines the moment when intracranial pressure is about to change (before the cisterna manga is injected into the mouse) as time of 0. At the same time, the ICP data recorded before time 0 is removed. According to the injection dose label, the ICP monitoring values were divided into different data segments to compare the differences in intracranial pressure response under different injection doses.
S3. Calculate the mean value

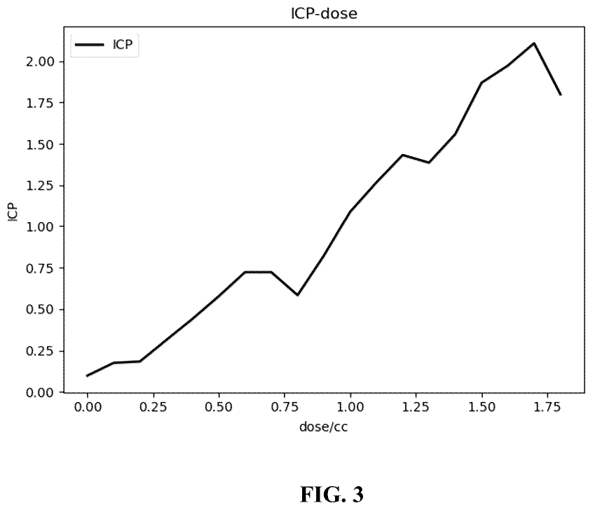

The ICP data at different stages were averaged. The result was taken as the ICP value at the current injection dose. The curve of ICP values at different injection doses was plotted as shown in FIG. 3.

The results showed that injection of saline from the cisterna manga induced an increase in intracranial pressure in rats. This is because when saline is injected, it directly affects the flow and volume of cerebrospinal fluid. The increased volume of fluid in the cranium in turn increases the pressure on the ventricular system, which is manifested as an increase in intracranial pressure. At the same time, there is a compensatory mechanism in the cranium that reduces the increase in intracranial pressure through a variety of means, such as cerebrospinal fluid circulation, cerebral blood flow, etc., so that there is a decreasing phase in the ICP monitoring curve. However, the compensatory capacity of the cranial brain is limited, and with the increase of the injected dose, it will still lead to the elevation of ICP.

### (2) Spectral Intensity Data Processing

### \textcircled{1} Background Light Denoising

Based on the wavelength-background spectral intensity data obtained for 7 min, the following measures were taken to reduce the experimental errors: i. In order to prevent the instability of the electronic components when the spectrometer was turned on, which led to the instability of the obtained data, the data of the first 2 min should be rounded off; ii. The resolution of the spectrometer used was 0.21 nm, and the three columns of spectral values at a specific wavelength and at a specific wavelength±0.21 nm were selected. From the three columns of spectral data at each wavelength of 700 nm, 725 nm, 750 nm, 775 nm, 800 nm, respectively, find the minimum value, and average the three minimum values to get the noise value at that wavelength.

The incident laser intensity at each wavelength detected is subtracted from the noise value at that wavelength separately. The average value is taken as the incident laser energy at that wavelength. The transmitted laser intensity at each detected wavelength is subtracted from the noise value at that wavelength, and the average value is taken as the transmitted laser energy at that wavelength. The average value is taken as the transmitted laser energy at that wavelength.

### \textcircled{2} Calculation of Absorbance

Absorbance was calculated using the formula:

$$A = {log}_{10} \frac{{\overline{I}}_{t} ( λ )}{{\overline{I}}_{o} ( λ )}$$

A is the absorbance, I̅ₜ̅(λ) denotes the energy of transmitted light, I̅ₒ̅(λ) A denotes the energy of the incident laser.

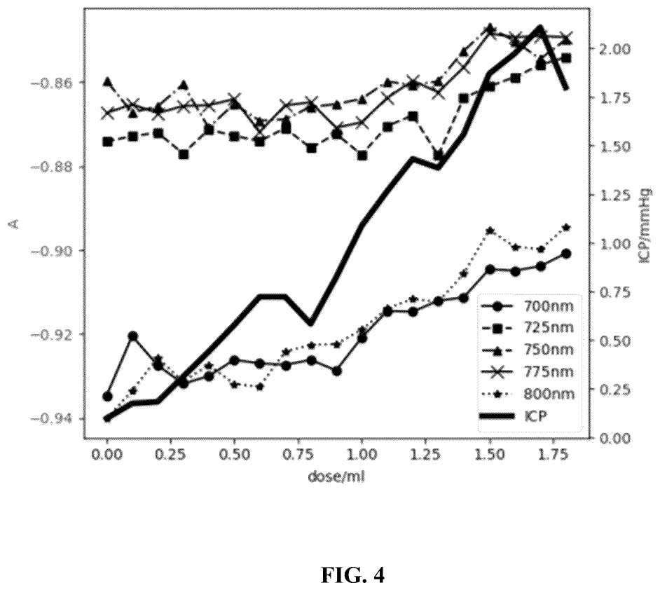

The results are shown in FIG. 4, which shows that the same substance attenuates the laser to different degrees when irradiated with different wavelengths. The results show that the waveforms at wavelengths of 700 nm and 800 nm can reflect the trend of positive correlation between the absorbance and the injected dose. While, the other three waveforms showed irregular changes.

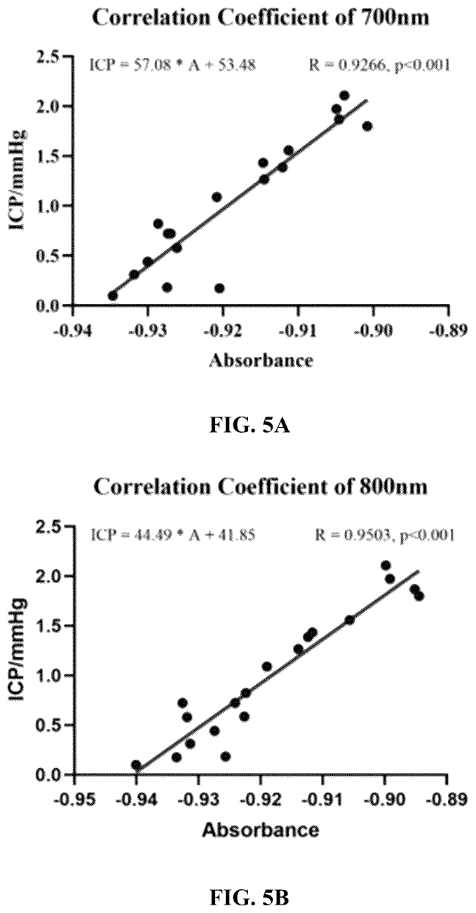

By analyzing the data of brain absorbance and intracranial pressure at 700 nm, 725 nm, 750 nm, 775 nm and 800 nm wavelength lasers. The results showed better correlation at 700 nm and 800 nm wavelengths. Afterwards, the relationship between absorbance A and ICP at 700 nm and 800 nm wavelengths was plotted by data fitting, and the relationship equation was also derived. The results are shown in FIG. 5.

FIG. 5 (A) shows the relationship between absorbance A and ICP at 700 nm. FIG. 5 (B) shows the relationship between absorbance A and ICP at 800 nm.

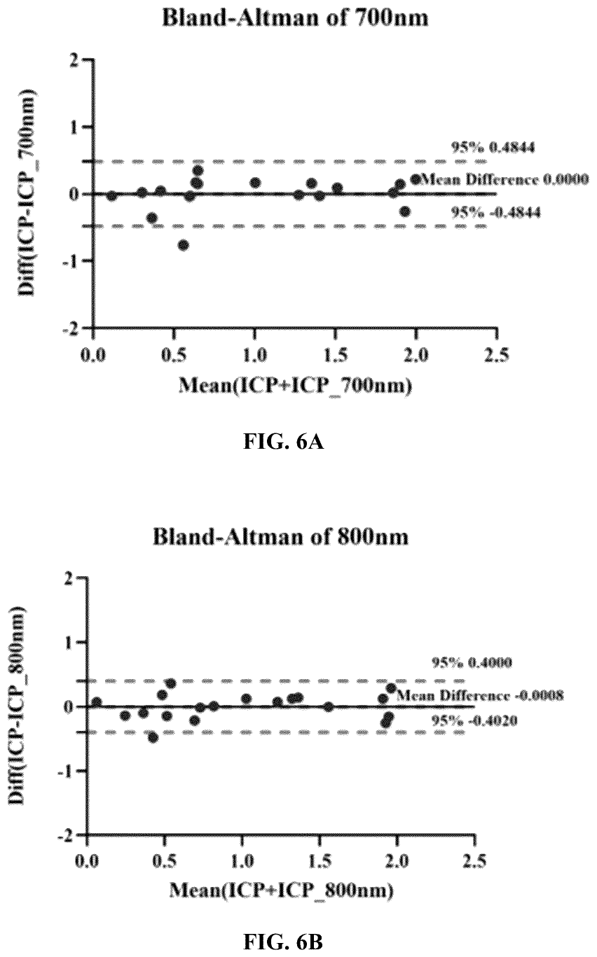

The ICP values obtained from the 2 fitted equations were then analyzed separately for consistency with the ICP values obtained through invasive lateral ventricular monitoring in Example 1. The results are shown in FIG. 6. Part (A) of FIG. 6 shows the consistency analysis between the ICP values of the fitted equations and the ICP values monitored by invasive lateral ventricle at 700 nm wavelength. Part (B) of FIG. 6 shows the agreement analysis between the fitted equation ICP values at 800 nm and the ICP values of invasive lateral ventricle monitoring. The results show that the two parameters are in high agreement and the agreement is better at 800 nm than 700 nm.

Therefore, the present invention adopts one of the above-described in-vivo non-invasive intracranial pressure monitoring devices and methods based on changes in meningeal absorbance. Compared with the traditional invasive intracranial pressure monitoring technique, the non-invasive monitoring method is capable of monitoring the patient's ICP in a non-invasive manner, which has the advantages of safety, low risk of infection, and continuous dynamic monitoring. It can greatly reduce the pain of patients in the process of monitoring ICP. It also has the advantages of low cost, high sensitivity, fast response time and no electromagnetic interference.

Finally, it should be noted that the above embodiments are only used to illustrate the technical solution of the present invention and not to limit it. Although the present invention is described in detail with reference to the preferred embodiments. The person of ordinary skill in the field should understand that the technical solution of the present invention can still be modified or equivalent replacement. And these modifications or equivalent substitutions cannot make the modified technical solution depart from the spirit and scope of the technical solution of the present invention.

What is claimed is:

1. An in vivo non-invasive intracranial pressure monitoring device based on meningeal absorbance changes, which is characterized in that: it comprises a signal excitation module, a spectral data acquisition module, a data processing module, and the signal excitation module comprises a laser and an optical parametric oscillator. The spectral data acquisition module includes a spectrometer and its supporting data acquisition software. Several mirrors are arranged between the output of the laser and the input of the optical parametric oscillator to adjust the optical path. The output end of the optical parametric oscillator is successively set up with a coaxial lens group and a fiber bundle, and the optical fiber beam outlet is connected to the monitoring object. The probe of the spectrometer is in contact with the monitoring object, and the probe is in the same plane as the optical fiber beam outlet.

2. A monitoring method for an in-vivo noninvasive intracranial pressure monitoring device based on changes in meningeal absorbance as claimed in claim 1. It is characterized in that it comprises the following steps:
S1. Pretreatment and fixation of monitoring objects.
S2. Detect background spectral intensity: Turn on the spectrometer and place the spectrometer probe at the irradiation site of the subject's skull. The spectrometer continuously records the background spectral intensity in real time for 7 min and obtains the background spectral intensity data that changes with wavelength and time.
S3. Detect the intensity of the incident laser: turn on the laser and preheat the optical parametric oscillator. The spectrometer probe is placed at the irradiation site of the subject's skull. The spectrometer probe is on the same straight line as the beam outlet. With 1 s as a cycle, the probe of the spectrometer is irradiated twice with a near-infrared pulsed laser of a certain wavelength in a cycle. Record the intensity of the incident laser for 10 cycles.
S4. Detect the intensity of the transmitted laser: keep the spectrometer probe in the same plane as the beam outlet. The optical end of the fiber beam is irradiated with a certain wavelength of near-infrared pulsed laser to monitor the irradiation site of the subject's skull. Turn on the spectrometer and place the spectrometer probe at the skull probe site of the monitored subject. The intensity data of the transmitted laser is collected every 0.1 s.
S5. Data processing and absorbance calculations.

3. Monitoring method of in vivo non-invasive intracranial pressure monitoring device based on meningeal absorbance change as claimed in claim 2. It is characterized in that: the pretreatment and fixation in the step S1 comprise anesthesia, removing and disinfecting the hair of the middle part of the skull of the monitoring subject, the monitoring subject lying supine, the body position is upright, and the head is horizontal with the body axis.

4. Monitoring method of in vivo non-invasive intracranial pressure monitoring device based on meningeal absorbance change as claimed in claim 2. It is characterized in that: the irradiation site is the midpoint of the anterior fontanelle.

5. Monitoring method of in vivo non-invasive intracranial pressure monitoring device based on meningeal absorbance change as claimed in claim 4. It is characterized in that, the detection site in the step S3 is that any point within the radius of 0˜3 cm with the irradiation site as the center of the circle.

6. Monitoring method of in vivo non-invasive intracranial pressure monitoring device based on meningeal absorbance change as claimed in claim 2. It is characterized in that: the near-infrared pulse laser of a certain wavelength is one of 700 nm, 725 nm, 750 nm, 775 nm, 800 nm.

7. In vivo non-invasive intracranial pressure monitoring method based on meningeal absorbance change as claimed in claim 2. It is characterized in that, the data processing and absorbance calculation in the step S5 comprise the following steps:
S5-1. The noise value is calculated according to the background spectral intensity data obtained in step S2 as a function of wavelength and time. Discard the data for the first 2 minutes. According to the resolution of the spectrometer and a certain wavelength when detecting the intensity of the incident laser and the transmitted laser. Find the minimum value in the three columns of background spectral intensity data of a certain wavelength, a certain wavelength±resolution. The average of the three minimum values is taken as the noise value.
S5-2. Calculate the incident laser energy. The intensity of the incident laser detected in step S3 is subtracted from the noise values, respectively. The average value is calculated as the incident laser energy.
S5-3, Calculate the transmitted laser energy. The transmitted laser intensities detected in step S4 are subtracted from the noise values, respectively. The average value is calculated as the transmitted laser energy.
S5-4. Absorbance calculation, absorbance is calculated as follows:
$$A = {log}_{10} \frac{{\overline{I}}_{t} ( λ )}{{\overline{I}}_{o} ( λ )}$$
A is the absorbance, I̅ₜ̅(λ) denotes the energy of transmitted light, I̅ₒ̅(λ) denotes the energy of the incident laser.
